(19) United States
(12) Patent Application Publication
(10) Pub. No.: US 2011/0301418 A1
(43) Pub. Date: Dec. 8, 2011

(54) Catheter Based 3-D Defocusing Imaging

(76) Inventors: Mortez Gharib, Altadena, CA (US); Jian Lu, San Gabriel, CA (US); David Jeon, Pasadena, CA (US)

(21) Appl. No.: 13/087,202

(22) Filed: Apr. 14, 2011

Related U.S. Application Data
(60) Provisional application No. 61/325,917, filed on Apr. 20, 2010.

(51) Int. Cl. A61B 1/07

(52) U.S. Cl. 600/181

(57) ABSTRACT

A catheter based defocusing imaging system for 3-D tomography reconstruction of endovascular features of interest is disclosed. Without limitation, target sites for imaging include heart valves, calcified heart valves, calcium plastered valve on the heart valve or plaque on the inner wall of the blood vessel of a patient.

### RELATED APPLICATIONS

The present filing claims the benefit of each of U.S. Patent Application Ser. No. 61/325,917 filed Apr. 20, 2010, entitled “Catheter Based 3-D Defocusing Imaging,” which application is incorporated herein by reference in its entirety.

### FIELD OF THE INVENTION

The present invention relates to a catheter imaging system and, more specifically, to a catheter imaging system that uses defocusing principles.

### BACKGROUND

Heart valve replacement is a major treatment for heart diseases with defective valves. Computerized Axial Tomography (CAT) scanning is a commonly used pre-operation tool to generate cross-sectional views of the heart valve. However, during the operation, it is still critical for doctors to know the real-time three-dimensional (3-D) tomography of the calcium plaster that has been pushed back to avoid damaging the valve and to properly position the implant valve. In addition, 3-D tomography can provide valuable information during diagnostics and treatment of blood vessel diseases caused by progressive accumulation of plaque on the inner walls. While 2-D axial views of internal organs can be obtained by imaging balloon catheters, obtaining 3-D tomography during surgeries still remains a challenge.

It is well known that a mask with two small apertures in an optical lens system will generate two defocusing images from a point source away from the focal plane of the system. The depth position (Z) of the point source can then be reconstructed by measuring the spacing between the two images. And the planar positions (X, Y) are measured from the center of its defocusing image pattern. Willert and Gharib implemented this concept by using three small apertures to generate more constrained image patterns, significantly reducing the ambiguities during 3-D reconstruction. Willert C. E. and Gharib M., 1992, Three-dimensional particle imaging with a single camera, Exp. Fluids 12 353-358.

### SUMMARY OF THE INVENTION

Commonly owned US Patent Publication No. 2010/0094138 published Apr. 15, 2010 discloses a system with hardware elements in common with one variation of the present system. However, the '138 application system is enabled for plaque depth imaging by analyzing the intensity of reflected laser light. The devices of the present system include features that enable defocusing imaging.

Accordingly, methods and device are provided to obtain 3-D tomography, for example, of the heart valve or the inner wall of blood vessels of a patient by characterizing the defocusing image patterns generated from the surface of the region of interest. The catheter based 3-D imaging system comprises, for example, an optical fiber bundle coupled with a conical mirror/reflector (mirror or reflector) or a rotating prism/mirror, a light projection system, a lens coupled with an aperture mask (optionally a 3-hole mask with or without central aperture), and a camera (CCD/diode/photo cell).

In one variation, the conical mirror/reflector (or a rotating prism/mirror) is held at the front end by a holder at the center of the fiber bundle. The light projection system may comprise a plurality of LEDs on the edge of the conical mirror/reflector such that tissues surrounding a transparent balloon are illuminated. During imaging, features (including many separated dark dots) in the tissues are used as markers to label the internal organ.

Alternatively, the calcified valve (or post balloon-catheter calcium plaster on the heart valve) or the plaque on the vessel wall can be labeled by projected laser dots. In this case, a small laser beam transmitted by one or more of the optical fibers in the optical bundle will generate a dot pattern after being transmitted through a diffractive optical element (DOE). The laser dot pattern may then be directed to the surface of the region of interest by another small conical mirror/reflector which is concentric to the outer conical one. In such a light projection system, a small band of clear window is provided in the outer mirror/reflector to allow the laser dots to go through and be projected onto the tissue.

Images of the features in the illuminated tissue or projected laser dot pattern on the surface are reflected back to the optical fiber bundle, and then transmitted through the lens and apertures to the camera (CCD/diode/photo cell). Using a mask with three or more defocusing apertures, a corresponding number of defocusing images forming a pattern (e.g., triangle, rectangle/square, etc.) similar to the configuration on the aperture mask will be generated from each marker (feature or laser dot). Therefore, the depth position as well as planar positions of each marker can be resolved by measuring its corresponding defocusing patterns. As such, 3-D tomography of the entire calcium plaster on the heart valve or plaque on the vessel wall can be reconstructed from 3-D locations of many markers.

Using the conical mirror arrangement, the reconstruction of the 3-D tomography does not require multiple scans of the region of interest because the illuminated features or the projected laser dots can label the entire field of view (e.g., a 360 degree band), avoiding damaging to the tissues as well as complexities in image processing.

If a relatively larger central aperture is added to the mask, a clear 2-D image of the object is captured in conjunction with the defocusing image patterns. An optical filter can be placed on the central aperture to separate the 2-D image. Features of the object in the 2-D image can be used to resolve camera Pose by existing methods (such as structure from motion). Accordingly, the teaching of US Patent Application Publication No. 2008/0278570 (i.e., CIT Number 4819), US 2009/0295908 (e.g., using a blue and red off-axis aperture pair to obtain camera pose) or PCT/US2010/057532 (e.g., with its multi-determination Pose methodology or other hardware features), each incorporated herein by reference in its entirety, may be employed.

In instances where the subject devices employ a mirror or prism system that is rotated to acquire images, determining camera Pose is important to enable the combining of the plurality of image frames obtained. The optical element may be rotatable within the catheter and/or balloon. Alternatively, they may be fixed and rotate with the catheter. The latter approach offers device simplicity, but may require more robust feature detection to “knit” images together based on markers (again anatomical features or laser dots) recognized between frames—be they adjacent or otherwise.

When employing the teaching of the '570 application (particularly those in connection with FIGS. 2G and 2H and associated text, in which a central aperture larger than offset smaller apertures is used to image a full image of an object, and the smaller apertures pass superimposed defocused dots) as applied in the present invention, the 2-D images obtained may picture no more than a section of the interior surface of the balloon. Especially when it may be difficult to image marker features beyond the balloon/blood or balloon/tissue barrier, the balloon itself may be of use when it includes marker features.

These features comprise an array of dots, or any sort of patterned printing on or in the balloon. Such patterning may be applied by pad printing, laser marking, etching or otherwise. The array or pattern may be black, or of a color selected to coordinate with a region (e.g., Red, Green or Blue in a commercially available CMOS or CCD sensor with a Bayer filter). Such color coding may be useful in reducing signal noise. For example, when a red laser is used for defocusing imaging (e.g., to transmit through blood), blue dots illuminated by a blue LED may offer optimal color separation in connection with an off the shelf sensor.

Patent Application Serial No. PCT/US2010/057532 teaches such a sub-selection strategy to reduce signal noise. However, the implementation above differs considerably in that the two different channels are not used in conjunction with each other to capture distinguishable image doublets for, variously, determining each of camera pose and 3-D surface information. Rather, one channel (in this non-limiting example—blue) is used for 2-D imaging to determine camera Pose and another channel (in this non-limiting example—red) to acquire reflected red laser point doublets, triplets, etc. for the purpose of determining 3-D surface information. Of course, third and even more color channels may be employed for 3-D determination if additional color light sources are used that are coordinated therewith or a broader-spectrum (e.g., white) light source is employed. Note, however, that color coding in either of the apertures or the sensor may not be necessary if we use more than one sensor (or sensor portion) and each is associated with one aperture.

When rotating the imaging device, different sections of the patterned balloon are imaged (i.e., captured by the imaging device). Used as reference features from frame-to-frame (note that these may be adjacent, sequentially taken frames or there may be skips, reversals, off-axis images, etc.) a map of camera position over time can be formed to allow combination/aggregation of the 3-D information resolved from the laser points captured through the defocusing apertures. Such an approach is indeed useful given that determining accurate catheter position from a remote location is both difficult and generally unreliable due to catheter wind-up and torsional “whipping” as well as axial compressibility. In this regard, the Pose approach may find utility outside of the field of 3-D imaging by defocusing and, as such, may be independently claimed in a generic sense. A non-limiting set of examples applications include use in/with: Structure From Motion (SFM), Scale Invariant Feature Transform (SIFT) and Speeded Up Robust Features (SURF) processes.

Multiple inventive aspects are disclosed herein. These aspects include the subject devices, programming associated with or running the same, kits in which they are included, and methods of use and manufacture. More detailed discussion is presented in connection with the figures below.

### BRIEF DESCRIPTION OF THE FIGURES

The figures provided herein are not necessarily drawn to scale, with some components and features exaggerated for clarity. Variations of the invention from the embodiments pictured are contemplated. Accordingly, depiction of aspects and elements of the invention in the figures are not intended to limit the scope of the invention.

FIG. 1 illustrates a catheter based 3-D imaging system with illuminated features serving as markers;

FIG. 2 illustrates a catheter baser 3-D imaging system with projected laser dots labeling the field of view;

FIGS. 3A-3F show alternative catheter prism/mirror arrangements;

FIG. 4A and 4B show a balloon section (open and closed, respectively) as may be utilized with any of the catheter body variations;

FIG. 5 is a block diagram showing the components of a data processing system embodiment; and

FIG. 6 is an illustration showing computer program product embodiments.

### DETAILED DESCRIPTION

Various exemplary embodiments of the aspects of the invention are described below. Reference is made to these examples in a non-limiting sense. They are provided to illustrate more broadly applicable aspects of the present invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. All such modifications are intended to be within the scope of the claims made herein.

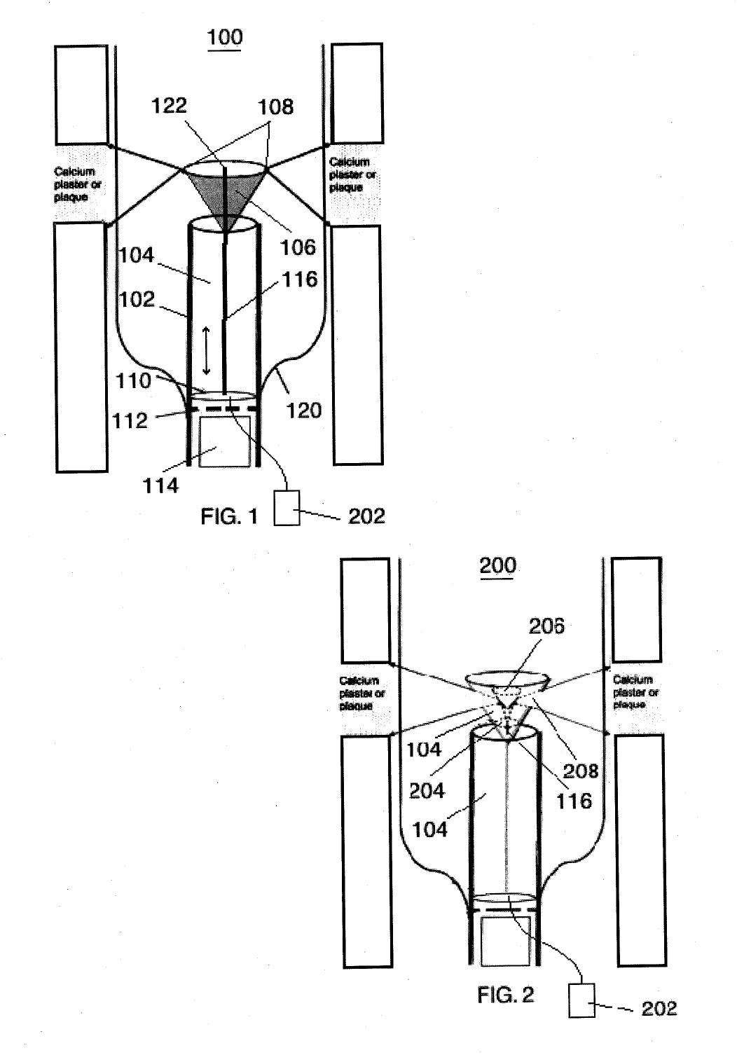

FIG. 1 illustrates a catheter based 3-D imaging system 100 with illuminated target features serving as markers. System 100 includes an elongate catheter body 102 housing an optical fiber bundle 104 coupled with a conical mirror/reflector 106, a light projection system by way of a plurality of LEDs 108, a lens 110 in association with an aperture mask 112, and a camera 114 (CCD/diode/photo cell). The lens/mask/camera may be housed within the catheter body (as shown) or connected outside by the fiber optic bundle through a typical optical adapter (not shown).

Using the conical mirror, the imaging zone subtends 360 degrees. For such purposes, mirror 106 may be supported at or through its apex by a holder 116 at the center of the fiber optic bundle 108. The holder 116 can be independent or connected with a guide wire of the catheter. For greater imaging range without repositioning the optional balloon 120, these optical components (i.e., at least the mirror, holder and fiber optic bundle) are capable of moving axially within the catheter body 102 as indicated by the double arrow. So-employed, multiple 360 degree bands that have been imaged can be “stitched” together or otherwise combined to yield a larger field. It should be noted that the conical mirror need not be perfectly conical, but can be substantially conical so as to cover those minor variations in shape that one of ordinary skill in the art would deem negligible for the purpose of imaging each side of the catheter.

In the variation in FIG. 1, the entire optical bundle 104 may be used for passing reflected light to the sensor. Otherwise, holder 116 may be hollow to define a lumen 122 to permit a central portion of the fiber optic to be used as a 2-D camera for visual observation or carrying out pose determinations according to the teachings of the above-referenced '580 patent application publication. As noted above, when a central imaging tract and associated central mask 112 aperture is employed, it may be desirable to utilize different wavelengths of light with associated filters on the apertures to distinguish between the central aperture and offset, smaller defocusing apertures. As such, two or more different color LEDs may be employed for illumination. Similarly, different polarized light sources and coordinated polarization filters may be employed.

The variation 200 in FIG. 2 differs primarily from that in FIG. 1 as described above in that it employs a portion of the optical bundle 104 to deliver light for projecting a pattern upon the target surface and includes associated mirror features for such purposes. In one example an LED or laser source 202 transmits through one or more of the optical fibers in the optical bundle 104 to form a dot pattern after passing through a diffractive optical element (DOE) 204. The laser dot pattern is shown to be directed to the surface to be imaged by another small conical mirror/reflector 206 which is concentric to the outer conical mirror 104. A small band of clear window(s) 208 is provided in the outer mirror/reflector to allow the laser dots to go through and be projected onto the tissue.

In yet another variation, the hardware in FIG. 1 may be employed in similar fashion to the system in FIG. 2 for projecting a pattern on the target surface by using the fiber optic bundle to transmit several narrow band laser beam dots around its center (generated by optional source device 202), which are then reflected by the conical mirror 106 and the transmitted through the (optional) transparent balloon.

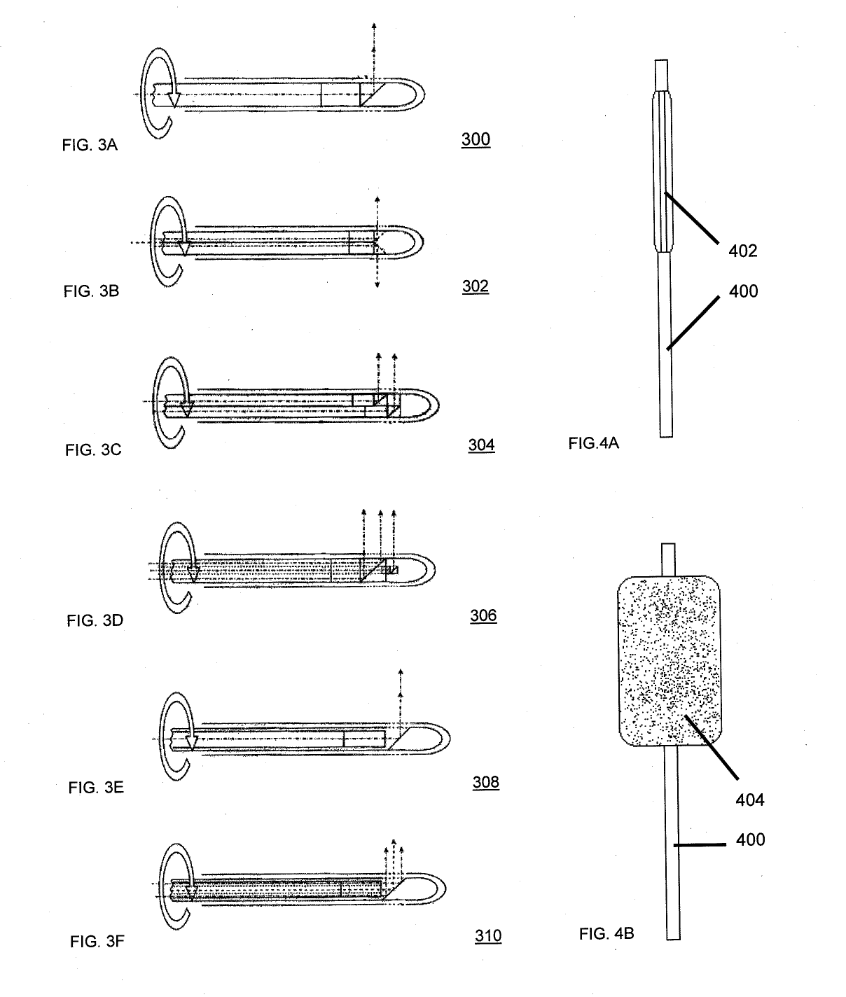

FIGS. 3A-3F show alternative catheter prism/mirror arrangements as may be employed in the present invention. Notably, U.S. Patent Publication No. 2005/0251116, incorporated by reference in its entirety, discloses various embodiments incorporating one or more prisms or mirrors coupled with a mechanical rotation device to obtain images of surrounding tissues from all angles. Since the aforementioned conical mirror embodiments of the present invention can reflect the laser beam to all angles of the surrounding tissues as well as direct the images from all angles back to the fiber bundle, they need not incorporate rotating mechanical elements.

Especially in instances in which miniaturization is key (e.g., imaging calcific lesions in distal coronary arteries or the neurovasculature), the conical mirror embodiments may be preferred. However, where space is not at such a premium (e.g., in imaging heart valves and other larger structures) the teachings of the '116 application may be utilized in conjunction with the other teachings herein. Namely, any of the six primary architectures disclosed and described therein and represented in FIGS. 3A-3F as embodiments 300, 302, 304, 306, 308 and 310 may all be employed as sub-components in embodiments of the present invention.

To do so, the fiber optics are coupled to a mask and sensor arrangement resembling that in FIGS. 1 and 2 (with or without the use of an optical adapter and/or laser for feature projection). Further contemplated modification includes provision of a balloon patterned with a marker array. Such a balloon 400 is illustrated prior to inflation in FIG. 4A. To maintain a minimal profile during physician or technician tracking to a site, the balloon may incorporate a number of folds 402 as common to coronary artery balloons and the like. The balloon is shown deployed/inflated in FIG. 4B, with the marker array 404 now evident. As an alternative to a balloon (as sometimes the case in endovascular devices) a braid-based self expanding or manipulable-braid “balloon” (see e.g. U.S. Pat. Nos. 4,650,466; 5,071,407; 5,222,971; 5,527,282; 5,496,277; 5,928,260; 6,344,048; and US Published Application No. 2005/0119684, each of which are incorporated by reference herein in their entirety) may instead be used. With the crossing wires and intersection points inherent to the braid matrix, a marker pattern with slight inherent variation is provided.

However configured, the “balloon” may be applied to any of the architectures in FIGS. 3A-3F as well as others using conventional techniques. In such embodiments, the relation of the balloon to the catheter body may be fixed. In this case, the prism(s)/mirror(s) may rotate, optionally together, with the fiber optic bundle. Otherwise, the catheter and optical components may rotate together with the balloon (and/or translate) with respect the balloon. Enabling the latter approach is within the level of skill in the art by incorporating one or more rotational and/or translational valves/wipers in the design (not shown). Furthermore, any additional structural details of the catheter body subcomponents pictured in FIGS. 3A-3F can be appreciated by reference to the incorporated '116 application.

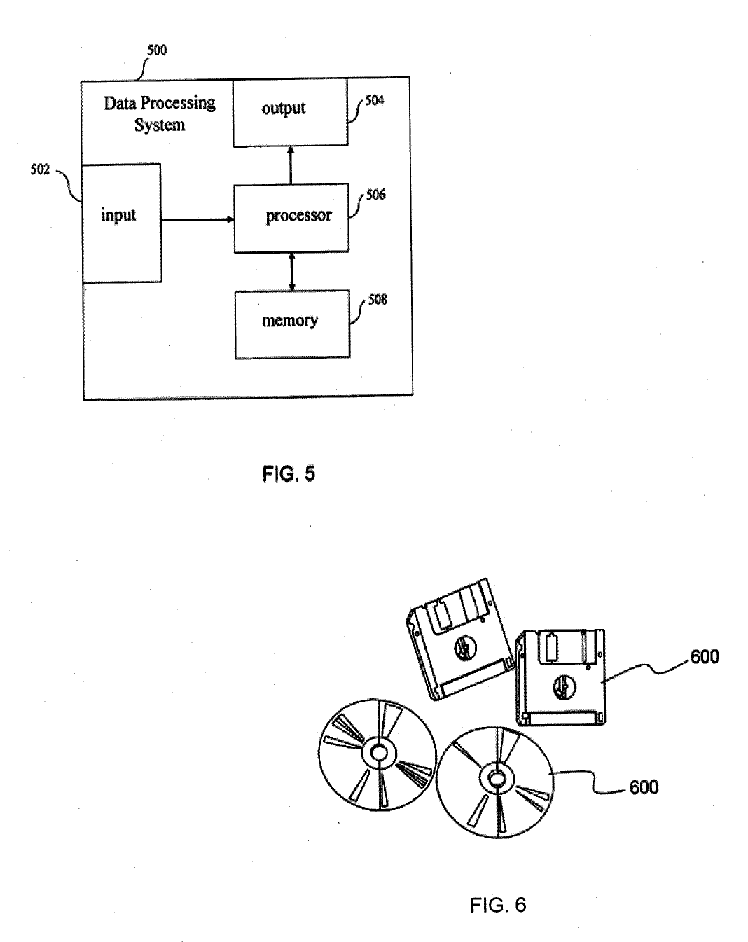

In addition, one skilled in the art can appreciate that the present invention also comprises a data processing system for executing the method of the present invention, as previously mentioned. A block diagram depicting the components of an embodiment of an image processing system of the present invention is provided in FIG. 5. The image processing system 500 comprises an input 502 for receiving information from at least one sensor for use in detecting image intensity of the non-coherent light captured by the sensor. Note that the input 502 may include multiple “ports.” Typically, input is received from at least one sensor, non-limiting examples of which include video image sensors. An output 504 is connected with the processor for providing information regarding the intensity profile of the image to other systems in order that a network of computer systems may serve as an image processing system. Output may also be provided to other devices or other programs; e.g., to other software modules, for use therein. The input 502 and the output 504 are both coupled with a processor 506, which may be a general-purpose computer processor or a specialized processor designed specifically for use with the present invention. The processor 506 is coupled with a memory 508 to permit storage of data and software that are to be manipulated by commands to the processor 506.

The present invention also comprises a computer program product. An illustrative diagram of a computer program product embodying the present invention is depicted in FIG. 6. The computer program product 600 is depicted as an optical disk such as a CD or DVD. However, as mentioned previously, the computer program product generally represents computer-readable instruction means stored on any compatible computer-readable medium. The term “instruction means” as used herein generally indicates a set of operations to be performed on a computer, and may represent pieces of a whole program or individual, separable, software modules. Non-limiting examples of “instruction means” include computer program code (source or object code) and “hard-coded” electronics (i.e. computer operations coded into a computer chip). The “instruction means” may be stored in the memory of a computer or on a computer-readable medium such as a floppy disk, a CD-ROM, and a flash drive.

### Variations

The subject methods may also include each of the physician activities associated with device positioning and use in imaging. Further, it is contemplated that any optional feature of the inventive variations described may be set forth and claimed independently, or in combination with any one or more of the features described herein. Reference to a singular item, includes the possibility that there is a plurality of the same items present. More specifically, as used herein and in the appended claims, the singular forms “a,” “an,” “said,” and “the” include plural referents unless specifically stated otherwise. In other words, use of the articles allow for “at least one” of the subject item in the description above as well as the claims below. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in the claims shall allow for the inclusion of any additional element irrespective of whether a given number of elements are enumerated in the claim, or the addition of a feature could be regarded as transforming the nature of an element set forth in the claims. Except as specifically defined herein, all technical and scientific terms used herein are to be given as broad a commonly understood meaning as possible while maintaining claim validity. Furthermore, any element in a claim that does not explicitly state “means for” performing a specified function, or “step for” performing a specific function, is not to be interpreted as a “means” or “step” clause as specified in 35 U.S.C. Section 112, Paragraph 6. In particular, the use of “step of” or “act of” in the claims herein is not intended to invoke the provisions of 35 U.S.C. 112, Paragraph 6.

The breadth of the present invention is not to be limited to the examples provided and/or the subject specification, but rather only by the scope of the claim language. All references cited are incorporated by reference in their entirety. Although the foregoing invention has been described in detail for purposes of clarity of understanding, it is contemplated that certain modifications may be practiced within the scope of the appended claims.

1. A catheter based defocusing imaging system comprising:
a fiber optic bundle received within an elongate catheter body;
at least one of a mirror or a prism positioned to pass light to or from the fiber optic bundle at an angle; and
the fiber optic connected to pass light to a sensor through an aperture mask including a plurality of apertures offset for defocusing imaging.

2. The system of claim 1, further comprising a central aperture in the mask for 2D imaging of a scene.

3. The system of claim 2, wherein the central aperture is coded to receive a signal different than the offset apertures.

4. The system of claim 3, wherein the central aperture passes light generated by at least one LED.

5. The system of claim 1, further comprising at least one additional aperture for determining camera pose.

6. The system of claim 1, wherein the offset apertures for defocusing imaging are coded to receive red light to allow imaging through blood.

7. The system of claim 6, wherein a laser produces the red light.

8. The system of claim 6, wherein the fiber optic bundle includes a projecting portion and a receiving portion,
the projecting portion configured to project the red light; and
the receiving portion configured to receive a reflected image signal of the projected light.

9. The system of claim 1, where the fiber optic bundle includes a projecting portion and a receiving portion,
the projecting portion configured to project light onto a material layer surface; and
the receiving portion configured to receive a reflected image signal of the projected light from the material layer surface.

10. The system of claim 1, further comprising a catheter balloon.

11. The system of claim 10, wherein the balloon includes an array of marker features.

12. The system of claim 10, wherein the catheter body is configured to move axially within the balloon catheter.

13. The system of claim 10, wherein the catheter body is configured to rotate within the balloon catheter.

14. The system of claim 13, wherein the fiber optic bundle and the at least one mirror or prism is configured to rotate within the catheter body.

15. The device of claim 10, including a mirror held by a holder portion near a center of the fiber optic bundle.

16. The device of claim 10, including a mirror that is at least substantially conical in shape, and positioned such that an apex of the mirror is located adjacent a terminus of the fiber optic bundle.

17. The system of claim 16, where in the mirror includes a central bore for forward observation.

18. The system of claim 16, further comprising an inner substantially conical mirror positioned within the outer mirror, the outer mirror including a clear band around a circumference of the outer mirror.

19. The system of claim 1, wherein the angle is about 90 degrees.

20. A method for catheter based 3-D imaging, comprising:
positioning an imaging catheter within a subject adjacent a target surface;
projecting light onto the surface through at least some blood;
receiving, through the blood, a reflected image signal of the projected light from the surface;
transmitting the reflected image signal through a fiber optical bundle in the catheter;
capturing a portion of the reflected image signal with a sensor masked by a plurality of apertures, wherein at least one of the apertures is offset from a central axis of the catheter; and
determining 3-D information for the surface by comparison of the captured portion of the reflected image signal.

21. The method of claim 21, wherein a conical mirror is used so that the captured image signals cover 360 degrees around the surface, and no camera pose determination is performed.

22. The method of claim 21, further comprising:
rotating at least one of a prism or mirror associated with the catheter;
capturing a plurality of image signal frames around the surface;
capturing marker image frames associated with a balloon portion of the catheter around the balloon; and
determining camera pose from the imaged marker array to aggregate at least some of the plurality of frames.
